(19) United States
(12) Patent Application Publication
(10) Pub. No.: US 2022/0372246 A1
(43) Pub. Date: Nov. 24, 2022

(54) PROCESS FOR PREPARING LIQUID OVERBASED METAL CARBOXYLATES, MIXED METAL STABILIZERS CONTAINING SAME, AND STABILIZED HALOGEN-CONTAINING POLYMERS THEREWITH

(71) Applicant: AM Stabilizers Corporation, Valparaiso, IN (US)

(72) Inventors: Nicholas A. Kruse, Valparaiso, IN (US); Laura K. Engerer, Valparaiso, IN (US)

(73) Assignee: AM Stabilizers Corporation, Valparaiso, IN (US)

(21) Appl. No.: 17/326,591

(22) Filed: May 21, 2021

(51) Int. Cl. C08K 5/138; C09K 15/08; C07C 51/41

(52) U.S. Cl. CPC C08K 5/138; C09K 15/08; C07C 51/412

(57) ABSTRACT

A process for producing liquid overbased alkali or alkaline earth metal carboxylates is disclosed wherein a para-cumyl phenol is used as a promoter of the process and the carboxylates produced are used as stabilizers for PVC.

### FIELD OF THE INVENTION

The present invention relates to a process for producing liquid overbased alkali or alkaline earth metal carboxylates, particularly barium carboxylates. Mixed metal stabilizers containing the overbased metal carboxylates are used as stabilizers for halogen-containing polymers such as polyvinyl chloride (PVC).

### BACKGROUND OF THE INVENTION

The preparation of overbased calcium or barium salts of carboxylic acids with alkyl phenols is well-known in the patent and industrial literature. The use of these overbased metal salts in halogen-containing polymers is also described in the literature and patents. Furthermore, the use of an alkyl phenol as a promoter in the manufacture of the overbased metal salts is well known.

However, due to recent legislation, primarily in Europe and Asia, with the accompanying impact on U.S. suppliers, there exists a need for an alkyl phenol-free overbased metal carboxylate. Also, environmental concerns with existing polymer stabilizers have stimulated interest in alternative stabilizers for the replacement of heavy metal stabilizers.

In particular, REACH regulations of the European Union have been adopted to improve the protection of human health and the environment from the risk that can be posed by chemicals. REACH stands for Registration, Evaluation, Authorization and Restriction of Chemicals and was entered into force on 1 Jun. 2007. REACH regulations have had an impact on most companies, not only across the EU, but also upon US manufacturers and suppliers in a wide range of companies across many sectors.

Due to REACH and other recent legislation, with the accompanying impact on US suppliers, there exists a need for an alkyl phenol-free overbased metal carboxylate. This invention is directed to satisfying this need, developing new environmentally acceptable PVC stabilizers that prevent polymer degradation and other changes during processing, and providing tangible benefits to the manufacture of useful articles.

### SUMMARY OF THE INVENTION

The present invention relates to a process for preparing a liquid overbased alkali or alkaline earth metal salt of carboxylic acid. The process involves reacting a mixture of the metal base and a carboxylic acid with an excess of metal base to carboxylic acid, and carbonating the reaction mixture to produce the overbased metal carbonate. It has been found that a p-cumyl phenol provided during carbonation of the reaction mixture produces desirable overbased alkali or alkaline earth metal salts having high levels of basicity, for example 20 to 40% barium or calcium. The p-cumyl phenol performs as well as an alkyl phenol as a promoter of the reaction to produce overbased metal salts under typical commercial preparation conditions.

Para-cumyl phenol has an aryl alkylene group in the para position of the phenol. Other names for this compound include p-(a,a′-dimethylbenzyl) phenol or 4-(2-phenylisopropyl) phenol. In other words, more broadly it may be referred to as p-phenylalkylene phenol. Preferably, the alkylene chain is about 1 to 6 carbon atoms, as exemplified by the isopropyl group. Therefore, in contrast to the alkyl phenol that heretofore was considered necessary to obtain a promoter for the reaction, a phenylalkylene group in the para position of the phenol has been used.

Thus, the method of this invention allows for the production of overbased alkaline metal carbonates without alkyl phenols. Moreover, compliance with regulations such as REACH that list alkyl phenols as undesirable can be met.

While this invention is not limited to a theoretical understanding of its benefits or processes, it is believed that, in the past, the alkyl group of the p-alkylphenols of the prior art was considered essential for the phenol to operate as a promoter in the production of the liquid overbased metal salts of carboxylic acid. However, it has been found that the phenylalkylene structure of the p-cumyl phenol enables the commercial performance advantages which have heretofore been achieved with the use of an alkyl phenol as a promoter. The performance advantages associated with the PVC use of overbased barium salts of the para-cumyl phenol include low plate-out, excellent color-hold, long-term heat stability performance, compatibility with stabilizer components, etc. In particular, PVC compositions which employ the overbased metal carboxylate of this invention are at least equivalent to those produced according to prior art techniques without the risks that may be posed by alkyl phenols.

The advantages, benefits and further understanding of this invention will be apparent with reference to the following detailed description and preferred embodiments.

### DETAILED DESCRIPTION OF THE INVENTION AND PREFERRED EMBODIMENTS

A. LIQUID OVERBASED ALKALI OR ALKALINE EARTH METAL SALTS OF PARA-CUMYL PHENOL/CARBOXYLIC ACID.

The present invention relates to a shelf-stable liquid overbased alkali or alkaline earth metal salt of a para-cumyl phenol and carboxylic acid. In a broader context, the para-cumyl phenol is a p-aralkylenephenol. The alkylene chain is about 1 to 6 carbon atoms., and the aryl is phenyl. These liquid salts are referred to herein sometimes as “cumyl phenate/carboxylate” because both the para-cumyl phenol and carboxylic acid enter into the reaction to produce shelf-stable liquids containing an alkaline earth metal carbonate such as calcium or barium carbonate, and a mixture of a metal para-cumyl phenate and carboxylate (hereinafter “para-cumyl phenate/carboxylate”). These liquids are referred to sometimes hereinafter more simply as “overbased alkali or alkaline earth metal salt(s)”, “overbased metal salt(s)”, or “overbased alkaline earth metal para-cumyl phenate/carbonate(s)”. Liquid overbased calcium and barium salts, in a preferred form of the invention, are essentially free of alkylphenol. The process for preparing a shelf-stable liquid of an overbased alkaline earth metal salt of a para-cumyl phenol/carboxylic acid involves reacting the alkaline earth metal base and the acid with an equivalent ratio of metal base to the combination of the para-cumyl phenol and acid being greater than 1:1 to make a basic product in the presence of a liquid hydrocarbon. An aliphatic alcohol may be employed in the reaction. The mixture is acidified, preferably by carbonation, and water is removed from the reaction product to obtain a shelf-stable liquid overbased alkaline earth metal salt.

This invention is predicated in part upon providing during carbonation a para-cumyl phenol which reacts at commercial rates as a promoter to produce the overbased metal salts having up to about 40% by weight, usually about 20 to 40% by weight, of the overbased calcium or barium metal. Up to the discoveries made in accordance with this invention it was not considered possible to make in any practical commercial operation, a highly overbased barium carboxylate/carbonate that was free of an alkyl phenol, for example, that may be filtered at commercial or practical rates.

The fatty acid of the overbased liquid salt is generally a C₁₂-C₂₂ fatty acid, including, for example, lauric, pyristic, palmitic, stearic, and behenic, among the saturated fatty acids. Unsaturated fatty acids include palmitoleic, oleic, linoleic, and linolenic. Among these fatty acids, oleic is presently preferred in preparing the overbased liquid carboxylates. The alkaline earth metal of the salt is selected from the group consisting of calcium, barium, magnesium, and strontium. Alkali metals include sodium, potassium, and lithium. For example, shelf-stable liquids of overbased calcium and barium oleates have been prepared. These overbased barium salts, for example, contain barium carbonate, barium oleate, barium cumyl phenate, a liquid hydrocarbon diluent, and an aliphatic alcohol.

B. PARA-CUMYL PHENOL

The para-cumyl phenol compound employed in this invention has an aryl group that enables the avoidance of the alkyl-phenol group that has been considered by REACH as posing a risk to human health and the environment.

In one preferred form of the invention, the shelf-stable liquid of an overbased barium salt of a para-cumyl phenol/fatty acid comprises a barium carbonate, a barium para-cumyl phenate/carboxylate of the fatty acid, a liquid hydrocarbon, and an aliphatic alcohol, with the liquid being free of an alkyl phenol which was required in prior practical commercial operations.

C. AMOUNTS OF REACTANTS AND CATALYSTS

The amount of alkali or alkaline earth metal base utilized in the preparation of basic salts is an amount which is more than one equivalent of the base per equivalent of the combined para-cumyl phenol/carboxylic acid or organic moiety, and more generally, will be an amount sufficient to provide at least three equivalents of the metal base per equivalent of the para-cumyl phenol. The alcohols that are used include any one of the various available substituted or unsubstituted alilphatic or cycloaliphatic alcohols containing from 1 to about 20 or more carbon atoms. The amount of the para-cumyl phenol and optionally the alcohol included in the mixture is not critical. The para-cumyl phenol promoter is included in the mixture to contribute to the utilization of the carbon dioxide gas during treatment of the mixture with the acidic gas. Generally, at least about 0.1 equivalent and preferably from about 0.05 to about 10 equivalents of the para-cumyl phenol (and the alcohol if present) per equivalent of a monocarboxylic acid is employed. Larger amounts, for example, up to about 20 to about 25 equivalents of alcohol and para-cumyl phenol may be used, especially in the case of lower molecular weight alcohols. Water, which may optionally also be present in the mixture, may be present as water added as such to the mixture, or the water may be present as “wet alcohol”, hydrates of the alkali or alkaline earth metal salts, or other types of chemically combined water with the metal salts.

In addition to the components described above, the reaction mixtures used to prepare the basic metal salts ordinarily will contain a diluent. Generally, any hydrocarbon diluent can be employed, and the choice of diluent is dependent in part on the intended use of the mixture. Most generally, the hydrocarbon diluent will be a nonvolatile diluent such as the various natural and synthetic oils of lubricating viscosity.

The amount of basic alkali or alkaline earth metal base utilized in the preparation of basic salts is an amount which is more than one equivalent of the base per equivalent of para-cumyl phenol and acid, and more generally, will be an amount sufficient to provide at least three equivalents of the metal base per equivalent of the acid and para-cumyl phenol. Larger amounts can be utilized to form more basic compounds, and the amount of the metal base included may be any amount up to that amount which is no longer effective to increase the proportion of metal in the product. When preparing the mixture, the amount of para-cumyl phenol and the optional alcohol included in the mixture is not critical except that the ratio of equivalents of monocarboxylic acid to cumyl phenol should be at least about 1.1:1, that is, the monocarboxylic acid is present in excess with respect to the para-cumyl phenol. The ratio of equivalents of the metal base of the combination of the other components in mixture should be greater than 1:1 in order to provide a basic product. More generally, the ratio of equivalents will be at least 3:1.

The step of carbonation involves treating the mixtures described above with CO₂ gas in the absence of free oxygen until the titratable basicity is determined using phenolphthalein. Generally, the titratable basicity is reduced to a base number below about 10. The mixing and carbonation steps of the present invention require no unusual operating conditions other than preferably the exclusion of free oxygen. The base, fatty acid, para-cumyl phenol, and liquid hydrocarbon are mixed, generally heated, and then treated with carbon dioxide as the acidic gas, and the mixture may be heated to a temperature which is sufficient to drive off some of the water contained in the mixture. The treatment of the mixture with the carbon dioxide preferably is conducted at elevated temperatures, and the range of temperatures used for this step may be any temperature above ambient temperature up to about 325° F., and more preferably from a temperature of about 130° F. to about 325° F. Higher temperatures may be used, but there is no apparent advantage in the use of such higher temperatures. Ordinarily, a temperature of about 130° F. to 325° F. is satisfactory.

D. HALOGEN-CONTAINING POLYMER

A halogen-containing polymer, such as a vinyl halide resin, most commonly stabilized with the basic metal salts of this invention is polyvinyl chloride. It is to be understood, however, that this invention is not limited to a particular vinyl halide resin such as polyvinyl chloride or its copolymers. Other halogen-containing resins which are employed and which illustrate the principles of this invention include chlorinated polyethylene, chlorosulfonated polyethylene, chlorinated polyvinyl chloride, and other vinyl halide resin types. Vinyl halide resin, as understood herein, and as appreciated in the art, is a common term and is adopted to define those resins or polymers usually derived by polymerization or copolymerization of vinyl monomers including vinyl chloride with or without other comonomers such as ethylene, propylene, vinyl acetate, vinyl ethers, vinylidene chloride, methacrylate, acrylates, styrene, etc. A simple case is the conversion of vinyl chloride H₂C═CHCl to polyvinyl chloride (CH₂CHCl—) wherein the halogen is bonded to the carbon atoms of the carbon chain of the polymer. Other examples of such vinyl halide resins would include vinylidene chloride polymers, vinyl chloride-vinyl ester copolymers, vinyl chloride-vinyl ether copolymers, vinyl chloride-vinylidene copolymers, vinyl chloride-propylene copolymers, chlorinated polyethylene, and the like. Of course, the vinyl halide commonly used in the industry is the chloride, although others such as bromide and fluoride may be used. Examples of the latter polymers include polyvinyl bromide, polyvinyl fluoride, and copolymers thereof.

Metal compound heat stabilizers of vinyl halide resin compositions are well known. These metal compounds serve to capture HCl liberated during heat processing of the vinyl halide resin composition into its final shape. The metal can be lead, cadmium, barium, calcium, zinc, strontium, bismuth, tin, or antimony, for example. The stabilizers are usually metal salts of a carboxylic acid, advantageously of a C₈-C₂₄ carbon chain link monocarboxylic acid such as lauric, oleic, stearic, octoic, or similar fatty acid salts. Mixed metal salts of such acids, and their preparation, are familiar to those skilled in the art to which this present invention pertains. Mixed metallic carboxylates involving calcium/zinc or barium/zinc blends alone and in combination with other stabilizers or additives such as beta-diketones, phosphite salts and phenolic antioxidants have been used. The metal stabilizer is a mixed metal salt of a carboxylic acid. Mixed metal salts of such acids, and their preparation, are also familiar to those skilled in the art to which this present invention pertains.

E. END USES FOR THE STABILIZERS

The liquid stabilizers or mixed metal stabilizers of this invention may be used in a number of end products. Examples include: wall covering, flooring (vinyl tile and inlay), medical devices, dip coating, chair mat, banner film, pigment dispersion, vinyl siding, piping, fuel additive, cosmetic, ceiling tile, roofing film, wear layer, play balls or toys, teethers, fencing, corrugated wall panels, dashboards, and shifter boots.

The following Examples illustrate the preparation of the shelf stable haze free liquids of the overbased salts in accordance with the method of the present invention, but these examples are not considered to be limiting the scope of this invention. Unless otherwise indicated in the following examples and elsewhere in the specification and claims, all parts and percentages are by weight, and all temperatures are in degrees fahrenheit.

### COMPARATIVE EXAMPLE I

The following ingredients and amounts were employed in this Comparative Example to demonstrate the normal procedure which has been employed to make a barium nonylphenate overbased salt.

| | Amount |
|---|---|
| Ingredients | (g) |
| Oleic Acid | 58.3 |
| 1418 Alcohol | 35 |
| 100 Neutral Oil | 101.5 |
| Nonyl Phenol | 30 |
| BaOH | 248 |

The 1418 alcohol is a commercially available mixture of aliphatic alcohols containing 14-18 carbon atoms, and the neutral oil is a mineral oil.

The oleic acid, oil, and alcohol ingredients were charged into a reaction vessel and mixed at room temperature while purging the vessel with nitrogen gas at 2 liters per minute. After a period of about 15-20 minutes, the mixture was heated while stirring to a temperature of about 133° F. At about 133° F., the BaOH was incrementally added in three separate additions of about 83, 81, and 84 grams each. At about 138° F., 2 drops of anti-foam were added to the reaction mixture. Thereafter, the reaction mixture was heated over about an hour to a temperature of about 240° F., whereupon the nonyl phenol was charged to the reaction mixture. After a period of about 10-15 minutes at a temperature of about 240° F., the reaction mixture was heated to about 265° F. During the course of the reaction, water was removed. After all of the nonyl phenol was charged, the nitrogen purge was stopped, and the mixture was carbonated with carbon dioxide at a rate of about 1 liter per minute for approximately 4.5 hours. 18 m Is of water were removed during the course of the reaction, and the resulting product was a filterable hot solution which titrated to 33.19% barium.

### EXAMPLE II

The objective of this example was to prepare an overbased barium para-cumyl phenate/monocarboxylate of this invention. This was achieved by replacing the nonyl phenol of Comparative Example 1 with an equivalent amount of para-cumyl phenol. For this purpose, the following ingredients and their actual amounts were employed.

| | Amount |
|---|---|
| Ingredients | (g) |
| Oleic Acid | 183.6 |
| 1418 Alcohol | 119.0 |
| 100 Neutral Oil | 482.8 |
| Para-cumyl phenol | 122.7 |
| BaOH | 814.3 |

The procedure of Comparative Example I was essentially followed, after substituting para-cumyl phenol for the nonyl phenol to make the overbased barium para-cumyl phenate/oleate carbonate salt. Approximately the same time table of Example I for mixing the reaction ingredients, heating and charging of the barium hydroxide and para-cumyl phenol were used at approximately the same temperatures. Except the para-cumyl phenol was added after warming in the oven to 80° C. The storage-stable liquid titrated to a barium content of about 32.88%.

### EXAMPLE III

In this Example, para-cumyl phenol was substituted for the nonyl phenol in Comparative Example I, and the following ingredients were employed.

| | Amount |
|---|---|
| Ingredients | (g) |
| Oleic Acid | 183.6 |
| 1418 Alcohol | 119.0 |
| 100 Neutral Oil | 101.6 |
| Para-cumyl phenol | 122.7 |
| BaOH | 814.3 |

Following the procedure of Comparative Example I, the reaction was conducted over similar reaction times and temperatures and, again, the para-cumyl-phenol was warmed in an oven at 80° C. The resulting storage-stable liquid was formed and filtered, titrated to barium in an amount of 32.9%.

### EXAMPLE IV

In this Example para-cumyl phenol was again substituted for nonyl phenol of Comparative Example I and the following ingredients were employed.

| | Actual |
|---|---|
| Ingredients | (g) |
| Oleic Acid | 195.5 |
| 1418 Alcohol | 127.5 |
| 100 Neutral Oil | 457.9 |
| Para-cumyl phenol | 103.1 |
| BaOH | 816.1 |

Following essentially the same procedure of Comparative Example I, liquid barium para-cumyl phenate/oleate carbonate was prepared that titrated to 33.25% barium.

### EXAMPLE V

The purpose of this example was to compare the stabilizing effectiveness of the metal salts of Examples 2-4 of this invention with the commercially available alkyl phenol stabilizer like Comparative Example 1. The standard technique of the industry was employed for this purpose and the results demonstrated equivalent stabilizing effectiveness in PVC for the metal salts of this invention upon comparison with the commercially available barium alkyl phenate stabilizers.

Commercially available 34% overbased barium nonyl phenate, sold as PLASTISTAB 2508 was formulated into a stabilizer composition as a control for the purpose of demonstrating heat-stabilizing effectiveness of the overbased metal salts of this invention as compounded stabilizers for PVC. Stabilizer compositions for each were formulated in a standard polyvinyl chloride (PVC) formulation at a level of four parts where the balance of the formulation included 100 parts of polyvinyl chloride. Each PVC formulation as milled at 365° F. for five minutes, and static heat stability was determined at 375° F. and 400° F. Over a period of about 40 minutes, stabilizing effectiveness of each composition was measured by color change. Color change was measured by colorimeter as an indication of yellowing and color values were determined by ASTM E313-73.

Both the color values and heat chip charts demonstrated the equivalent effectiveness of the basic alkaline earth metal salts of this invention by a comparison with a commercially available basic barium nonyl phenate.

The above description provides a disclosure of particular embodiments of the invention and is not intended for the purpose of limiting the same thereto. As such, the invention is not limited to only the above described embodiments, rather, it is recognized that one skilled in the art would understand alternative embodiments in view of the above description that fall within the scope of the invention.

What is claimed is:

1. A process for preparing a storage-stable liquid overbased alkali or alkaline earth metal salt comprising:
reacting a mixture of an alkali or alkaline earth metal base and a carboxylic acid with an equivalent ratio of metal base to carboxylic acid being greater than 1:1 in the presence of liquid hydrocarbon, and
carbonating the reaction mixture in the presence of a p-phenylalkylene phenol which acts as a promoter of the reaction during carbonation to produce the storage-stable liquid overbased alkali or alkaline earth metal salt.

2. The process of claim 1 wherein the carboxylic acid is an aliphatic or aromatic carboxylic acid and the phenylalkylene phenol has a lower alkylene chain of 1 to 6 carbon atoms.

3. The process of claim 2 wherein said carboxylic acid is a C₁₂-C₂₂ fatty acid,

4. The process of claim 3 wherein said fatty acid is oleic acid.

5. The process of claim 1 wherein said alkaline earth metal is selected from the group consisting of calcium, barium, magnesium, and strontium.

6. The process of claim 1 wherein the alkali metal is selected from the group consisting of sodium, potassium, and lithium.

7. The process of claim 1 wherein said alkaline earth metal is barium.

8. The process of claim 7 wherein the overbased salt is a barium oleate/cumylphenate/carbonate.

9. The process of claim 1 wherein the amount of alkali or alkaline earth metal in the salt is up to about 40% by weight.

10. The process of claim 9 wherein the amount of alkali or alkaline earth metal in the salt is about 25% to about 40% by weight.

11. The process of claim 1 wherein the reaction is conducted in the presence of alcohol.

12. A process for preparing a storage-stable liquid overbased barium salt comprising
reacting a barium hydroxide and a carboxylic acid with an equivalent ratio of barium hydroxide to carboxylic acid being greater than 1:1 in the presence of liquid hydrocarbon, carbonating the reaction mixture in the presence of a p-phenyalkylene phenol which acts as a promoter of the reaction during carbonation to produce the overbased barium salt and byproduct water, and
removing water from the reaction product to provide the storage-stable liquid overbased barium carboxylate/phenylalkylene phenate/carbonate.

13. The process of claim 12 wherein the p-phenylalkylene phenol is p-cumylphenol.

14. The process of claim 13 wherein said organic acid is an aliphatic or aromatic carboxylic acid.

15. The process of claim 14 wherein said carboxylic acid is a C₁₂-C₂₂ fatty acid.

16. The process of claim 15 wherein said fatty acid is oleic acid.

17. The process of claim 12 wherein the amount of alkali or alkaline earth metal in the salt is about up to 40% by weight.

18. The process of claim 13 wherein the reaction is conducted in the presence of alcohol.

19. The liquid overbased alkali or alkaline earth metal salt of the p-phenylalkylene phenol and carboxylic acid prepared in accordance with the process of claim 1.

20. The liquid overbased alkali or alkaline earth metal salt of a p-phenylalkylene phenol and carboxylic acid prepared in accordance with the process of claim 13.

21. A halogen-containing polymer composition comprising a halogen-containing polymer and a heat-stabilizing amount of a liquid overbased alkaline earth metal salt of a p-phenylalkylene phenate/carboxylate.

22. The halogen-containing polymer composition of claim 21 wherein the metal salt is a barium cumyl phenate/carboxylate.
